(19) United States
(12) Patent Application Publication
(10) Pub. No.: US 2009/0216063 A1
(43) Pub. Date: Aug. 27, 2009

(54) BIO-ABSORBABLE BRACHYTHERAPY STRANDS

(75) Inventors: Gary A. Lamoureux, Woodbury, CT (US); James Matons, Woodbury, CT (US)

Correspondence Address: FLIESLER MEYER LLP, 650 CALIFORNIA STREET, 14TH FLOOR, SAN FRANCISCO, CA, 94108, US

(73) Assignee: BIOCOMPATIBLES UK LIMITED, Farnham (GB)

(21) Appl. No.: 12/361,285

(22) Filed: Jan. 28, 2009

Related U.S. Application Data
(60) Provisional application No. 61/024,389, filed on Jan. 29, 2008.

(51) Int. Cl. A61M 36/12

(52) U.S. Cl. 600/7

(57) ABSTRACT

Provided herein are bio-absorbable strands for use in brachytherapy. In an embodiment, a plurality of discrete hollow bio-absorbable segments spaced apart from one another and encapsulated using a bio-absorbable material to form an elongated member configured to be implantable in patient tissue using a hollow needle. Each hollow bio-absorbable segment has a length, an outer periphery and an inner channel. Radioactive material is within at least a portion of the inner channel or coating at least a portion of the outer periphery of each hollow bio-absorbable segment. Contrast material is within at least a portion of the inner channel or coating at least a portion of the outer periphery of each hollow bio-absorbable segment.

### PRIORITY CLAIM

This application claims priority under 35 U.S.C. 119(e) to U.S. Provisional Patent Application No. 61/024,389, entitled “Bio-Absorbable Brachytherapy Strands,” filed Jan. 29, 2008, which is incorporate herein by reference.

### BACKGROUND

In interstitial radiation therapy, a tumor can be treated by temporarily or permanently placing small, radioactive seeds into or adjacent the tumor site. This can be accomplished by implanting loose seeds in the target tissue, or by implanting in the target tissue seeds that are connected to one another by a bio-absorbable material.

To implant loose seeds, an applicator device (e.g., a Mick™ applicator or the like) that includes a needle is often used. A stylet is initially fully extended through a bore in the needle and the needle is inserted into a patient in an area where a row of loose seeds are to be implanted. The stylet is then retracted from the needle, enabling a loose seed from a magazine to enter the bore of the needle. The stylet is then pushed against the loose seed, forcing the seed through the bore of needle and into the target tissue. After a first seed has been implanted, the needle is withdrawn from the patient's body by a particular distance so that a next seed to be implanted is spaced apart from the first seed. Then, the stylet is again retracted to enable the next seed from the magazine to be positioned for movement into the needle. The stylet is then advanced through the needle to force the next seed into the target tissue at a desired distance away from the first seed. This procedure is repeated for subsequent seed implants. Additional details of this implantation technique and the applicator used to perform this technique can be found in U.S. Pat. No. 5,860,909, which is incorporated herein by reference.

In the above technique, loose seeds are deposited in a track made by the needle. However, when the needle is withdrawn, there is a tendency for the seeds to migrate in that track resulting in improper distribution of the seeds. Additionally, after implantation, the loose seeds are dependent on the tissue itself to hold each individual seed in place. This may result in the loose seeds migrating over time away from the initial site of implantation. Such migration of seeds is undesirable from a clinical perspective, as this may lead to underdosing or overdosing of a tumor or other diseased tissue and/or exposure of healthy tissue to radiation. The loose seeds may also rotate or twist from the original orientation at which the seeds were implanted. This is also undesirable from a clinical perspective, because the radiation pattern of the seeds may be directional, thereby causing underdosing or overdosing of a tumor or other diseased tissue and/or exposure of healthy tissue to radiation. Further complicating the implantation of loose seeds is the fact that the seeds are small, because they need to fit in small bore needles to prevent excessive tissue damage. Due to their small size and high seed surface dose, the seeds are difficult to handle and to label, and can easily be lost. In addition, the above described technique for implantation of individual loose seeds is time consuming.

Because of the disadvantages of using loose seeds, many physicians prefer using elongated members (often referred to as strands) that contains multiple seeds spaced from one another at desired increments. Such strands are capable of being loaded into an introducer needle just prior to the implant procedure, or they may be pre-loaded into a needle. Implantation of strands is less time consuming than implanting loose seeds. Additionally, because the seeds in the strands are connected to one another by a bio-absorbable material, there is less of a tendency for the seeds to migrate and/or rotate after implantation.

There are numerous techniques for making strands that include multiple seeds. For example, such strands can be made using a bio-absorbable material, with the seeds and rigid teflon spacers between the seeds inserted into the material. Needles loaded with the seeds in the carrier bio-absorbable material are sterilized or autoclaved causing contraction of the carrier material and resulting in a rigid column of seeds and spacers. This technique was reported in “Ultrasonically Guided Transperineal Seed Implantation of the Prostate: Modification of the Technique and Qualitative Assessment of Implants” by Van't Riet, et al., International Journal of Radiation Oncology, Biology and Physics, Vol. 24, No. 3, pp. 555-558, 1992, which is incorporated herein by reference. Such rigid implants have many drawbacks, including not having the ability to flex with the tissue over the time that the bio-absorbable material dissolves. More specifically, as the tissue or glands shrink back to pre-operative size, and thus as the tissue recedes, a rigid elongated implant does not move with the tissue, but remain stationary relative to the patient. The final locations of the seeds relative to the tumor are thus not maintained and the dosage of the radioactive seeds does not meet the preoperative therapy plan. Accordingly, there is a desire to provide a strand of seeds that is capable of moving with tissue or glands as they shrink back to pre-operative size, thereby enabling the seeds to meet a preoperative therapy plan.

In another technique, disclosed in U.S. Pat. No. 5,460,592, which is incorporated herein by reference, seeds are held in a woven or braided bio-absorbable carrier such as a braided suture. The carrier with the seeds laced therein is then secured in place to form a suitable implant. This braided assembly exhibits many drawbacks, as and when the braided assembly is placed into the target tissue. The needle that carries the braided strand assembly must be blocked at the distal end to prevent body fluids from entering the lumen. If body fluid reaches the braided strand assembly while the assembly is still in the lumen of the needle, the braided assembly can swell and jam in the lumen. Because the assembly is made of a braided tubular material, it is difficult to push the assembly out of the needle. As the needle is withdrawn from the tumor, pressure on the proximal end of the braided strand assembly causes the braid to expand and jam inside the lumen of the needle. Finally, if the braided strand is successfully expelled from the needle, the relative spacing of the seeds may not be maintained, if the braided material has collapsed. Accordingly, there is also a desire to provide a strand of seeds that can be implanted without causing jamming of a needle, and that after implantation the strand maintain the desired spacing of the seeds.

It is also desirable for a strand of seeds to be echogenic, i.e., be visible using ultrasound imaging, so that the implant can be visualized during implantation and during post operative visits to a physician. Techniques have been developed for making the seeds themselves more echogenic. For example, U.S. Pat. No. 6,632,176 suggests that seeds can be roughened, shaped or otherwise treated to improve the ultrasound visibility of the seeds. However, it is desirable that an entire strand be visible, not just the seeds therein. It has been suggested that the particles of materials such as glass, silica, sand, clay, etc. be mixed in with the bio-absorbable material to make the strand assembly of seeds more visible to ultrasound. However, the additions of such particles may effect the integrity of the strand. Additionally, such particles may irritate tissue after the bio-absorbable material has been absorbed. Further, it may be desirable to simply minimize the volume of materials that are not going to be absorbed by the body. Also, because it may be difficult to control the distribution of such particle, strand including such particles may not be uniformly visible by ultrasound.

Another technique that has been suggested to increase the ultrasound visibility of a strand of seeds is to introduce air bubbles into the bio-absorbable material during the manufacture of the strand, since air is a strong reflector of ultrasound energy having an inherent impedance many times greater than body tissue. This can be accomplished during the cooling stage of a molding process used to produce the strand, as disclosed in U.S. patent application Ser. No. 10/035,083, filed May 8, 2003, which is incorporated herein by reference. More specifically, during the cooling stage, the mold is placed in a vacuum chamber and the air in the chamber is evacuated. This causes the entrapped air in the mold to come out of solution from the polymer, and as the mold cools, this air is entrapped within the cooling polymer in the form of minute bubbles suspended in the plastic. A potential problem with this technique, however, is the inability to control the placement and size of the air bubbles. Thus, a strand including such air bubbles may not be uniformly visible by ultrasound. Accordingly, there is also a desire to improve the ultrasound visibility of a strand of seeds.

Regardless of whether radioactive seeds are implanted loosely, or as part of a strand, such seeds typically include small metal housings, generally made of titanium or stainless steel, within which a radioactive material is sealed. Typically the only way to remove conventional radioactive seeds, after implantation, is through invasive surgery. Thus, such radioactive seeds are typically left within the patient indefinitely, even after the effective radiation dose has been delivered. The presence of these metallic seed housings may interfere with subsequent diagnostic X-rays or other imaging modalities, and may interfere with other treatment modalities, such as thermal ablation or external beam radiation. Additionally, such metallic housings can migrate to undesirable locations within the patient's body after implantation, while still effectively emitting therapeutic radiation and/or after the radioactive source has decayed.

### BRIEF SUMMARY

Provided herein are bio-absorbable strands for use in brachytherapy. In an embodiment, a plurality of discrete hollow bio-absorbable segments spaced apart from one another and encapsulated using a bio-absorbable material to form an elongated member configured to be implantable in patient tissue using a hollow needle. Each hollow bio-absorbable segment has a length, an outer periphery and an inner channel. Radioactive material is within at least a portion of the inner channel or coating at least a portion of the outer periphery of each hollow bio-absorbable segment. Contrast material is within at least a portion of the inner channel or coating at least a portion of the outer periphery of each hollow bio-absorbable segment.

This summary is not intended to be a complete description of the invention. Other and alternative features, aspects, objects and advantages of the invention can be obtained from a review of the specification, the figures, and the claims.

### BRIEF DESCRIPTION OF THE FIGURES

FIG. 1A illustrates a strand according to an embodiment of the present invention.

FIG. 1B is a cross-sectional view of the strand of FIG. 1A, along line 1B-1B.

FIG. 1C illustrates a strand according to an alternative embodiment of the present invention.

FIG. 1D illustrates that segments, of embodiments of the present invention, can be encapsulated between a pair of bio-absorbable half-shell members to form a strand.

FIG. 2A shows a side view of a helical segment, according to an embodiment of the present invention, which can be encapsulated to make one of the strands of FIGS. 1A-1D.

FIGS. 2B-2D are various cross sectional views of the segment shown in FIG. 2A.

FIG. 2E is used to illustrate how, in accordance with an embodiment, strings can be used to produce the segment shown in FIG. 2A.

FIG. 3 is an exemplary rotating structure that can be used to produce the segment shown in FIG. 2E.

FIG. 4 is a cross section of a strand formed using helical segments of FIG. 2A at a point where a helical segment includes radioactive material and contrast media.

FIG. 5 is an exemplary device that can be used to insert strands of the present invention into a patient.

### DETAILED DESCRIPTION

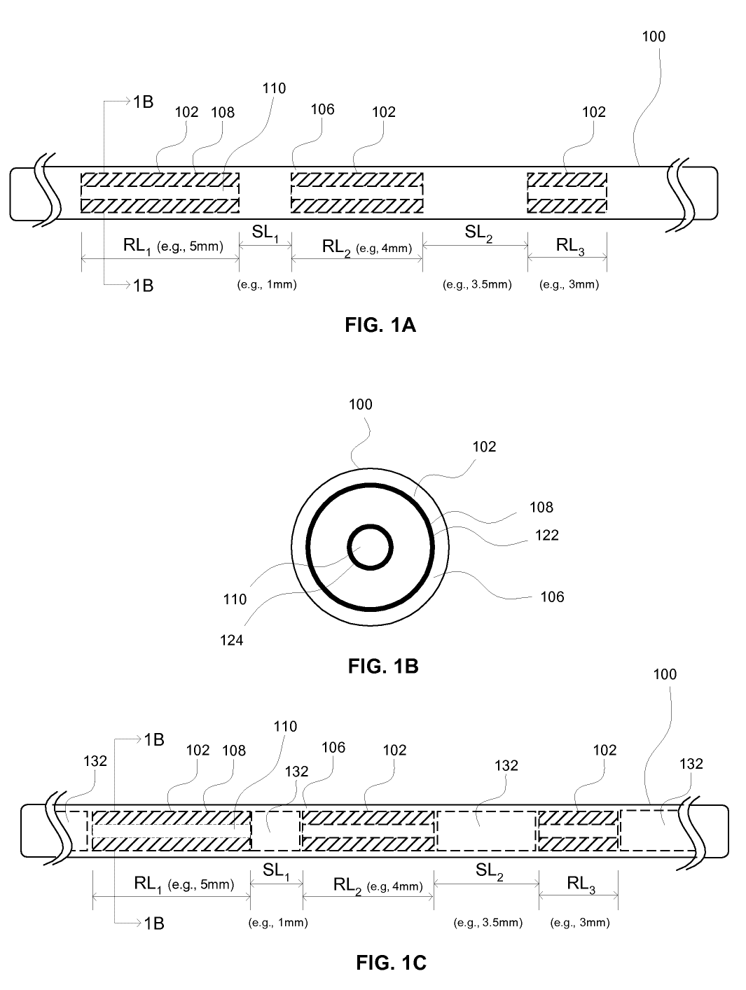

Disclosed herein are bio-absorbable strands that are especially useful for brachytherapy. Referring to FIG. 1A, a strand 100 according to an embodiment of the present invention is shown as including a plurality of discrete hollow bio-absorbable segments 102 spaced apart from one another and encapsulated (e.g., overmolded or pushed into a hollow tube) by a bio-absorbable material 106 to form an elongated member configured to be implantable in patient tissue using a hollow needle. FIG. 1B is a cross-sectional view of the strand 100 of FIG. 1A, along line 1B-1B.

Each hollow bio-absorbable segment 102 has a length (e.g., RL₁, RL₂ and RL₃ in FIG. 1A), an outer periphery 108 and an inner channel 110. In accordance with an embodiment, included within at least a portion of the inner channel 110 of each hollow bio-absorbable segment 102 is a contrast media 124, such as, but not limited to, a radiopaque material. Additionally, a radioactive material 122 coats at least a portion of the outer periphery 108 of each hollow bio-absorbable segment 102. Alternatively, the radioactive material is within at least a portion of the inner channel 110 of each hollow bio-absorbable segment 102, and the contrast media 124 coats at least a portion of the outer periphery 108 of each hollow bio-absorbable segment 102. It is also possible that both the radioactive material and contrast media coat the outer periphery, e.g., one above the other, or along different portions of the outer periphery 108. It is also possible that both the radioactive material and contrast media are included within the inner channel 110 of a segment 102, e.g., one above the other, or at different portions of the inner channel 110.

A benefit of embodiments of the present invention, over conventional strands that include radioactive seeds, is that the entire strand 100 is bio-absorbable. Accordingly, there are no non-bio-absorbable metallic or plastic seed housings that remain indefinitely in the patient's body. This is very useful where such seeds may undesirable migrate, such as in fatty tissue (e.g., in breast tissue) and collect at one location. Further, there is no need remove any materials (e.g., seed housings) from a patient's body, e.g., through surgery.

Typically, radioactive seeds used in brachytherapy are only available in predefined lengths. In contrast, in accordance with an embodiment of the present invention, the segments 102 that include (e.g., are coated with) radioactive material can be of any desired length. In accordance with an embodiment, the plurality of hollow bio-absorbable segments 102 (which have the contrast material within at least a portion of the inner channel and the radioactive material coating at least a portion of the outer periphery, or vice versa) have lengths that are in accordance with a treatment plan such that a length of one segment 102 can be different than a length of another segment 102. For example, referring to FIG. 1A, length RL₁ can be different than RL₂, which can be different than RL₃.

Additionally, or alternatively, the lengths of the plurality of spacings between segments 102 can be in accordance a treatment plan such that a length of one of the spacings can be different than a length of another one of the spacings. For example, spacing length SL₁ can be different than SL₂, which can be different than SL₃ (not labeled). The spacings can be achieved with or without the use of discrete spacers 132. More specifically, referring to FIG. 1C, the plurality of hollow bio-absorbable segments 102 can be spaced apart from one another by a plurality of discrete spacers 132, which can be used to maintain the spacings between segments 102. The spacers can have lengths SL₁, SL₂, etc., which can differ from one another, depending on a treatment plan.

The bio-absorbable hollow segments 102 can be manufactured using any known method, such as extrusion, casting, punch pressing, injection molding, compression molding blow molding, milling, etc. The bio-absorbable hollow segments 102 can be made of the same bio-absorbable material as the material encapsulating (e.g., used to overmold) the segments (and optional spacers 132) to form the strand 100. Alternatively, the encapsulating (e.g., overmolding) material can be a different bio-absorbable material than the material used to make the segments 102. For example, where the segments 102 (and optional spacers 132) are encapsulated by inserting them into a hollow tube to form a strand, the segments 102 and the hollow tube into which the segments are inserted, can be made of the same (or different) bio-absorbable material(s). Referring to FIG. 1D, in still another embodiment, the segments 102 (and optional spacers 132) can be encapsulated between a pair of bio-absorbable half-shell members 107a and 107b, and the half-shell members 107a and 107b can be fused or otherwise attached to one another to form a strand. Additional details of such half-shell members are disclosed in U.S. Pat. No. 7,244,226, which is incorporated herein by reference.

More generally, the strand 100 can be manufacture in various manners. For example, the strand 100 can be manufactured using a hollow tube or Vicryl “sock” by pushing the segments 102 and spacers 132 into the tube, or by a molding processes, such as, but not limited to, compression molding or injection molding. The bio-absorbable segments 102 can be of the same length, or of different lengths, if a preoperative therapeutic plan so specifies. Also, spacing between segments 102 (and thus, optional spacers 132) can be of the same length, or of different lengths, if the preoperative therapeutic plan so specifies. The segments 102 (and/or spacer 132) can be made available in the plurality of different lengths, or segments (and/or spacers 132) can be cut to their proper lengths.

Example types of bio-absorbable materials that can be used to produce the segments 102 (and/or spacers 132) include, but are not limited to, synthetic polymers and copolymers of glycolide and lactide, polydioxanone and the like. Such polymeric materials are more fully described in U.S. Pat. Nos. 3,565,869, 3,636,956, 4,052,988 and European Patent Publication No. 0030822, all of which are incorporated herein by reference. Specific examples of bio-absorbable polymeric materials that can be used to produce embodiments of the present invention are polymers made by ETHICON, Inc., of Somerville, N.J., under the trademarks “MONOCRYL” (polyglycoprone 25), “MAXON” (Glycolide and Trimethylene Carbonate), “VICRYL” (polyglactin 910, also known as PGA) and “PDS II” (polydioanone).

Other exemplary bio-absorbable materials include poly(glycolic acid) (PGA) and poly(-L-lactic acid) (PLLA), polyester amides of glycolic or lactic acids such as polymers and copolymers of glycolate and lactate, polydioxanone and the like, or combinations thereof. Such materials are more fully described in U.S. Pat. No. 5,460,592 which is hereby incorporated by reference. Further exemplary bio-absorbable polymers and polymer compositions that can be used in this invention are described in the following patents which are hereby incorporated by reference: U.S. Pat. No. 4,052,988 which discloses compositions comprising extruded and oriented filaments of polymers of p-dioxanone and 1,4-dioxepan-2-one; U.S. Pat. No. 3,839,297 which discloses compositions comprising poly[L(−)lactide-co-glycolide] suitable for use as absorbable sutures; U.S. Pat. No. 3,297,033 which discloses the use of compositions comprising polyglycolide homopolymers as absorbable sutures; U.S. Pat. No. 2,668,162 which discloses compositions comprising high molecular weight polymers of glycolide with lactide; U.S. Pat. No. 2,703,316 which discloses compositions comprising polymers of lactide and copolymers of lactide with glycolide; U.S. Pat. No. 2,758,987 which discloses compositions comprising optically active homopolymers of L(−) lactide i.e. poly L-Lactide; U.S. Pat. No. 3,636,956 which discloses compositions of copolymers of L(−) lactide and glycolide having utility as absorbable sutures; U.S. Pat. No. 4,141,087 which discloses synthetic absorbable crystalline isomorphic copolyoxylate polymers derived from mixtures of cyclic and linear diols; U.S. Pat. No. 4,441,496 which discloses copolymers of p-dioxanone and 2,5-morpholinediones; U.S. Pat. No. 4,452,973 which discloses poly(glycolic acid)/poly(oxyalkylene) ABA triblock copolymers; U.S. Pat. No. 4,510,295 which discloses polyesters of substituted benzoic acid, dihydric alcohols, and glycolide and/or lactide; U.S. Pat. No. 4,612,923 which discloses surgical devices fabricated from synthetic absorbable polymer containing absorbable glass filler; U.S. Pat. No. 4,646,741 which discloses a surgical fastener comprising a blend of copolymers of lactide, glycolide, and poly(p-dioxanone); U.S. Pat. No. 4,741,337 which discloses a surgical fastener made from a glycolide-rich blend of polymers; U.S. Pat. No. 4,916,209 which discloses bio-absorbable semi-crystalline depsipeptide polymers; U.S. Pat. No. 5,264,540 which discloses bio-absorbable aromatic polyanhydride polymers; and U.S. Pat. No. 4,689,424 which discloses radiation sterilizable absorbable polymers of dihydric alcohols. If desired, to further increase the mechanical stiffness of the molded embodiments of the present invention, bio-absorbable polymers and polymer compositions can include bio-absorbable fillers, such as those described in U.S. Pat. No. 5,521,280 (which is incorporated by reference) which discloses a composition of a bio-absorbable polymer and a filler comprising a poly(succinimide); and U.S. Pat. No. 4,473,670 (which is incorporated by reference) which discloses bio-absorbable polymers and a filler of finely divided sodium chloride or potassium chloride.

In accordance with an embodiment, the bio-absorbable material should preferably be absorbed in living tissue in a period of time of from about 70 to about 120 days, but can be manufactured to be absorbed anywhere in a range from 1 week to 1 year, depending on the therapeutic plan for a specific patient. In an embodiment, the bio-absorbable material is selected to absorb about when the half-life of the radioactive material is reached.

Exemplary radioactive materials that can be used in embodiments of the present invention can emit either singly or in some combination gamma rays, x-rays, positrons, beta particles, alpha particles, or Auger electrons. Any of a wide variety of radioactive materials employed for brachytherapy may be employed in this invention, including but not limited to radioisotopes such as I-125, I-131, Y-90, Re-186, Re-188, Pd-103, Ir-192, P-32 and the like, but may also consist of any other radioisotope with an acceptable half-life, toxicity, and energy level. Thus, the radioisotope may include a radioactive metal ion, such as radioisotopes of rhenium. Possible isotopes for use in this invention include, but are not limited to, Cu-62, Cu-64, Cu-67, Ru-97, Y-90, Rh-105, Pd-109, Re-186, Re-188, Au-199, Pb-203, Pb-211 and Bi-212. In certain embodiments, the radioactive material is bio-absorbable.

The radioactive material can include a bonding component suitable for covalent or non-covalent attachment to a substrate material (e.g., the outer periphery 108 or inner channel 110 of the segments 102). In an exemplary embodiment, bifunctional chelates are covalently or otherwise bonded to the substrate material, e.g., through an amine functional group bonded to the substrate material, which substrate material may include a siloxane coating, including an aliphatic hydrocyclosiloxane polymer coating, and the bifunctional chelate is then radiolabeled. A variety of bifunctional chelatcs can be employed; most involve metal ion binding to thiolate groups, and may also involve metal ion binding to amide, amine or carboxylate groups. Representative bifunctional chelates include ethylenediamine tetraacetic acid (EDTA), diethylenetetramine-pentaacedic acid (DTPA), chelates of diamide-dimercaptides (N2S2), and variations on the foregoing, such as chelating compounds incorporating N2S3, N2S4 and N3S3 or other combinations of sulfur- and nitrogen-containing groups forming metal binding sites, and metallothionine. It is also possible, and contemplated, that a substrate material will be employed to which metal ions may be directly bonded to the substrate material, in which case the substrate material may include an amine functional group bonded to the surface of the substrate material. As an alternative to chemical bonding, the radioisotopes can be attached to a surface (e.g., the outer periphery 108 or inner channel 110 of a segment 102) by other known techniques, such as spraying, deposition, electroplating, electroless plating, adsorption, and ion pairing.

The contrast material, within at least a portion of the inner channel 110, or coating at least a portion of the outer periphery 108, enables a physician to view where the segments 102 are implanted, and thus where radiation is being delivered. In an embodiment, contrast material is a radiopaque material that can be detected by X-rays and/or other imaging techniques. Exemplary radiopaque materials that can be used include iodixanol, sold under the trade names Visipaque and Acupaque, and iohexyl, sold under the trade names Omnipaque and Exypaque, which are Food and Drug Administration-approved iodine-containing radiopaque agents. Ethiodized oils, such as those sold under the trade names Lipiodol and Ethiodol, may also be employed. The foregoing are non-ionic, iodinated radiopaque agents. Other iodine-containing radiopaque agents include acetrizoate sodium, iobenzamic acid, iocarmic acid, iocetamic acid, iodamide, iodized oil, iodoalphionic acid, iodophthalein sodium, iodopyracet, ioglycamic acid, iomegiamic acid, iopamidol, iopanoic acid, iopentol, iophendylate, iophenoxic acid, iopromide, iopronic acid, iopydol, iopydone, iothalmic acid, iotrolan, ioversol, ioxaglic acid, ipodate, propyliodone and the like. Metal-containing contrast agents may also be employed, such as barium sulfate, which can be mixed with polymers such as polyurethane to increase radioopacity. Many of the iodine-containing radiopaque agents are water soluble, such as iodixanol and iohexyl, while other iodine-containing radiopaque agents are largely or wholly insoluble in water, though they may be soluble in other solvents. Metallic elements with suitable biocompatibility and radiopacity include titanium, zirconium, tantalum, barium, bismuth and platinum. The preferred organic elements for biocompatibility and radiopacity are bromine, iodine, barium, and bismuth. Tantalum and platinum are used as stent components and barium sulfate and bismuth trioxide are used as radiopaque enhancements for polymer catheters. In specific embodiments the contrast material is bio-absorbable.

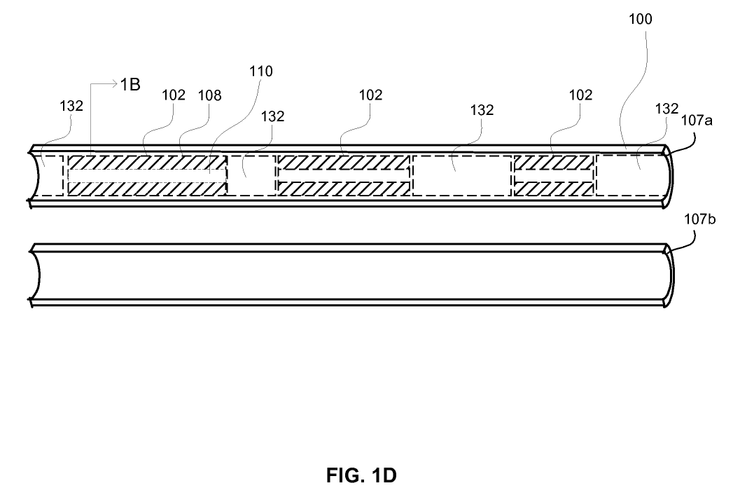

FIG. 2A shows a side view of a segment 102, according to an embodiment of the present invention. Three cross sectional views of the segment 102 are shown in FIGS. 2B, 2C and 2D. As can be seen from the cross sectional views, the segment 102 is made up of three strings 204 that twist about a hollow chamber 206 (i.e., the inner channel 110 in this embodiment). Because the three strings 204 twist about the hollow chamber 206, an outer surface 208 of the hollow chamber 206 is helical, and more specifically in this embodiment a triple helical. The segment includes an outer peripheral surface 210 (i.e., the outer periphery 108 in this embodiment) and an inner circumferential surface, with the inner circumferential surface of the segment being the outer surface of the hollow chamber 206. As shown in FIG. 2B, the inner circumferential surface includes three helical grooves 212₁, 212₂ and 212₃, and the outer circumferential surface 210 includes three helical grooves 214₁, 214₂ and 214₃, with each of the grooves being formed where the strings 204 meet one another. Because of its shape, the segment 102 shown in FIGS. 2A-2D may be referred to as a helical segment 102.

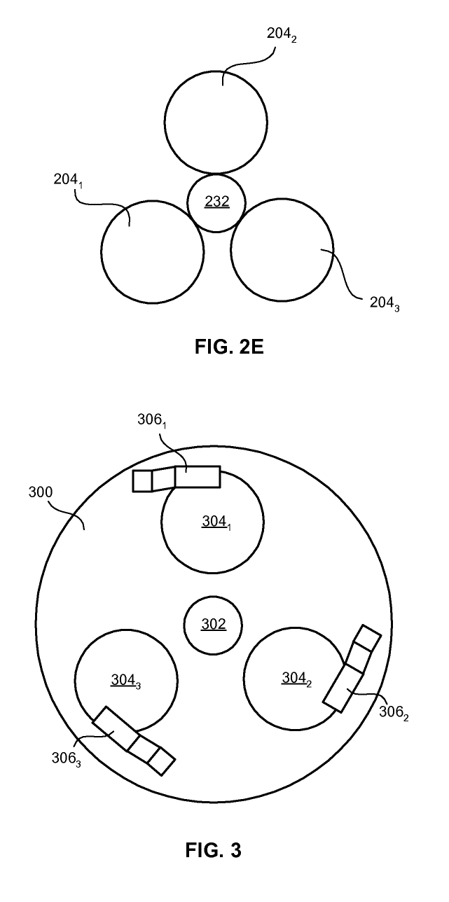

As was discussed above, included in at least a portion of the inner channel 206(110) of each hollow bio-absorbable helical segment 102 is a contrast media 124, such as, but not limited to, a radiopaque material. Additionally, a radioactive material 122 coats at least a portion of the outer periphery 210(108) of each hollow bio-absorbable helical segment 102. Alternatively, the radioactive material is within at least a portion of the inner channel 206(110) of each hollow bio-absorbable helical segment, and the contrast media 124 coats at least a portion of the outer periphery 210(108) of each hollow bio-absorbable segment. It is also possible that both the radioactive material and contrast media coat the outer periphery, e.g., one above the other, or an different portions of the outer periphery 210(108). It is also possible that both the radioactive material and contrast media are included within the inner channel 206(110) of a helical segment 102, e.g., one above the other, or at different portions of the inner channel 206(110). A cross section of a strand 100 formed using the helical segments 102, at a point where a helical segment includes radioactive material 122 and contrast media 124, is shown in FIG. 4. Where the helical segment 102 is used to form a spacer, there will be no radioactive material 122 or contrast media 124, but the cross section would look similar.

In accordance with an embodiment of the present invention, the strings 204 used to form the helical segments (or helical spacers) are made of a polymeric bio-absorbable material. In one specific embodiment, the strings 204 are lengths of suture material that can be purchased from ETHICON, Inc., of Somerville, N.J., under the trademark “MONOCRYL” (polyglycoprone 25). A list of other possible materials for the strings 104 are provided below. The diameter of each string is, for example, between 0.005 and 0.020 inches, with a preferably diameter of about 0.012 inches. However, other diameters are possible. Other exemplary bio-absorbable materials from which the strings can be made are discussed above.

In accordance with an embodiment of the present invention, the helical segment 102 is manufactured by twisting the three strings 204 around a fixed wire or mandrel that is coated with a mold release substance, such as silicon. The three strings 204 in their twisted arrangement are then heated, and then cooled, such that the strings 204 thermal set in the twisted configuration. The wire or mandrel is then pulled out of the center, leaving the a structure that is made up of three twisted strings of polymeric bio-absorbable material, with its hollow center having the triple helix outer surface 208. The structure is then cut to appropriate sizes, to produce bio-absorbable segments 102 and/or spacers 132. Because of their shape, such structures have improved ultrasound visibility. Like a tightly wound spring, such segments will be generally axially rigid and radially flexible. Accordingly, a strand that is made using such hollow segments should be generally axially rigid and radially flexible, which is desirable. Where spacers are used to separate the strands, the spacers can be solid spacers, or hollow spacers. Where the spacers are hollow, the spacers can have the same structure as the segment 102 shown in FIGS. 2A-2D, which is beneficial since spacers having such a structure are echogenic.

FIG. 2E, which is an end view of the three strings 204 prior to their twisting, shows that the three strings 204 can be initially evenly spaced around a wire or mandrel 232, with the centers of the strings 204 preferably being about 120 degrees apart from one another. Also shown in FIG. 2E is that a cross section of each string 104 can be generally circular, but this need not be the case.

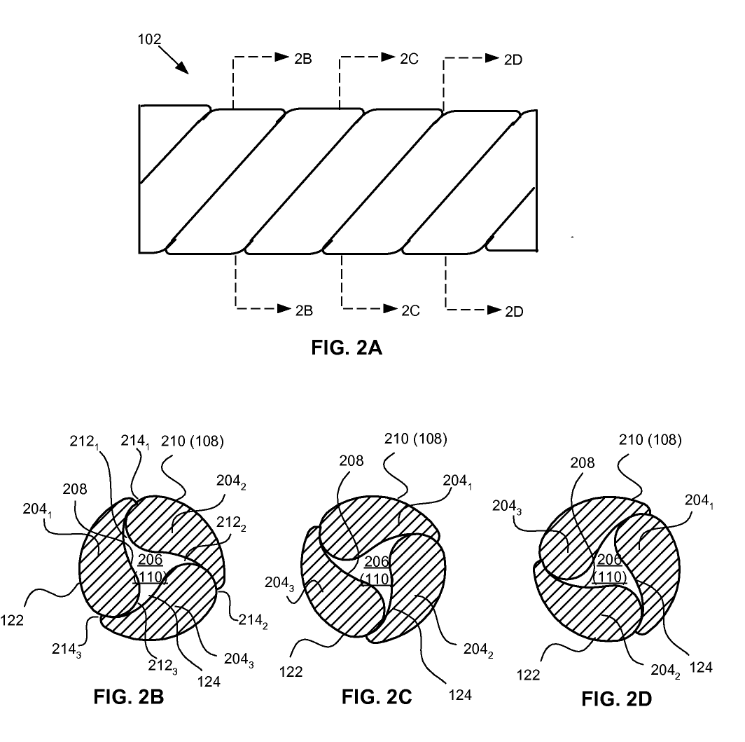

In a specific implementation, the wire or mandrel 232 is threaded or fed through a hole in the center of a rotating structure, and both longitudinal ends of the wire or mandrel 232 are fixedly attached (e.g., clamped) within a fixture, such that the wire or mandrel is pulled taut, and such that the rotating structure can rotate about the wire or mandrel. An exemplary rotating structure 300 that can be used is shown in FIG. 3. In addition to have a hole 302 in its center, the rotating structure 300 also includes three openings 304 that are about 120 degrees apart from one another and spaced around the hole 302. Each of these three openings 304 is configured to accept one of the three strings 204. A diameter of the rotating structure is, e.g., about 0.75 inches. The diameters of the center opening 302 and other openings 304 should be slightly greater than the wire/mandrel or stings to be placed through the openings.

The strings 204 are fixed (e.g., clamped) at one end of the fixture, in the arrangement shown in FIG. 2E. The other end of the strings 204 are fed through corresponding openings 304 in the rotating structure 300, shown in FIG. 3. Flat springs 306, or some other means, are used to hold the ends of the strings within the holes 306. Such springs 306 should allow for some slippage of the strings 204 when they shrink during heating, which is described below. Preferably about ten percent of each string 204 extends past the rotating structure 300 and hangs freely, so that the strings 204 do not release from the flat springs 304 when they are eventually heated and shrink. Once in this arrangement, the rotating structure 300 is turned in one direction (clockwise or counterclockwise) to thereby twist the strings 204 around the wire or mandrel 232. As the rotating structure 300 is turned, each string 204 twists around the wire or mandrel 202, causing the rotating structure 300 to be pulled toward the fixed ends of the strings 104.

In one embodiment, the wire or mandrel 232 has a diameter of about 0.007 inches, and each string 204 has an initial diameter of about 0.012 inches. With such dimensions, in accordance with an embodiment, the strings 204 are twisted around the wire or mandrel 232 such that the combined pitch of the strings is between 20 and 30 turns per inch, and preferably about 25 turns per inch. This would mean that each individual string 204 winds around the wire or mandrel about 6 to 10 times per inch, and preferably about 8 times per inch. This will result in the overall length of the twisted sting structure being about one-third of the original length of the strings 104. For example, if the strings 204 are initially 12 inches in length, the length of the structure made up of the twisted strings 204 will be about 4 inches.

After the strings 204 are twisted around the wire or mandrel 232 to achieve a desired pitch, the rotating structure 300 is then fixed in place, e.g., using another clamp, so that the strings 204 don't unwind. The entire fixture can then be placed in an oven or otherwise exposed to heat, to thereby heat the strings 204. Preferably, the twisted strings 204 are placed in the oven while the oven is at least 100 degrees F. lower than the desired temperature to which the strands will be exposed. This desired temperature, which is dependent on the material from which the strings 204 are made, is a temperature at which the strings 204 will shrink, but not melt. For example, if the strings 204 are made from polyglycoprone 25 (MONOCRYL™), then the strings 204 (and the fixture that holds the strings in place) should be placed in an oven when the oven is less than 360 degrees F., and then the oven should be raised to a temperature of about 460 degrees F. At this temperature, the strings 204 will shrink in diameter and length, forming tight spirals around the wire or mandrel. A small amount of fusion may occur between the strings 204, but this is not necessary. The flat springs 306 will allow the strings 204 to slip a little through their openings 304 in the structure 300, without releasing the strings 204.

The entire fixture, with the rotated strings 204 held in place, is then cooled. Once cooled, the strings 204 are thermo set in their tightly wound configuration. At that point, the strings 204 are released from the fixture, and the wire or mandrel 232 is removed, thereby leaving an elongated structure that is made up of tightly wound strings 204, with a hollow center chamber having an outer surface that is helical, and in this specific implementation a triple helix. This elongated structure is then cut into desired lengths of the segments 102 (and/or the spacers 132).

The inner diameter of the resulting segment 102 is dependent upon the diameter of the wire or mandrel 232 around which the strings 204 were wound. Thus, if the wire or mandrel had a diameter of 0.007 inches, then the inner diameter of the segment 102 (which defines the size of the channel 108) will be about 0.007 inches. The outer diameter of the segment 102 will be dependent on the diameter of the wire or mandrel 232 around which the strings 204 were wound, the diameter of each string 204, and the amount by which the strings shrink during the thermal setting process. Assuming the wire or mandrel 232 has a diameter of about 0.007 inches, and the diameter of each string 204 is about 0.012 inches, then the outer diameter of the segment 102 will be about 0.026 inches.

Ultrasound visibility is highly dependent upon the angular orientation of a surface with respect to the ultrasound inducer that is used for imaging. Generally, a smooth surface will act as a mirror, scattering ultrasound waves in a numerous directions unless the angle between the sound and the surface is very close to 90 degrees. Accordingly, if surfaces of a segment or spacer were relatively smooth, such surfaces would reflect ultrasound waves in a generally fan shaped conical pattern that spanned a large spatial angle, only giving a strong ultrasound reflections when imaged at an angle very close to 90 degrees. In contrast, the outer surface 208 of the hollow chamber 206 is helical, at least a portion of the surface 208 will likely be substantially 90 degrees from incoming ultrasound waves. Accordingly, if spacers are used to separate segments, it would be advantageous if the spacers has the structure described with reference to FIGS. 2A-2E, to avoid angular dependence of the reflected ultrasound.

While it is preferred that at least three strings 204 are used, it is also within the scope of the present invention that a single string 204, or two strings 204 be used. It is also within the scope of the present invention that more than three strings 204 may be used. Regardless of the number of strings 204, spacers can be made by twisting the strings 204 around a wire or mandrel, thermal setting the twisted string structure, and then removing the wire or mandrel, as was described above with reference to FIGS. 2 and 3. Changing the number of strings 204 used will simply change the number of helical grooves 212 in the inner circumferential surface (i.e., the outer surface of the hollow chamber) and the number of helical grooves 214 in the outer circumferential surface.

As mentioned above, the segments 102 of the present invention can be used to form strands, instead of using metallic radioactive seeds. Such a strand would include a plurality of segments 102 spaced apart from one another at desired intervals. These intervals can be selected to be any distance or combination of distances that are optimal for the treatment plan of a patient. The strand is preferably axially flexible such that it can be bent back upon itself in a circle without kinking. However, the strand preferably has sufficient column strength along its longitudinal axis so that the strand can be urged out of a hollow needle without the strand folding upon itself. The segments 102 of the present invention allow the stand to be axially rigid and radially flexible.

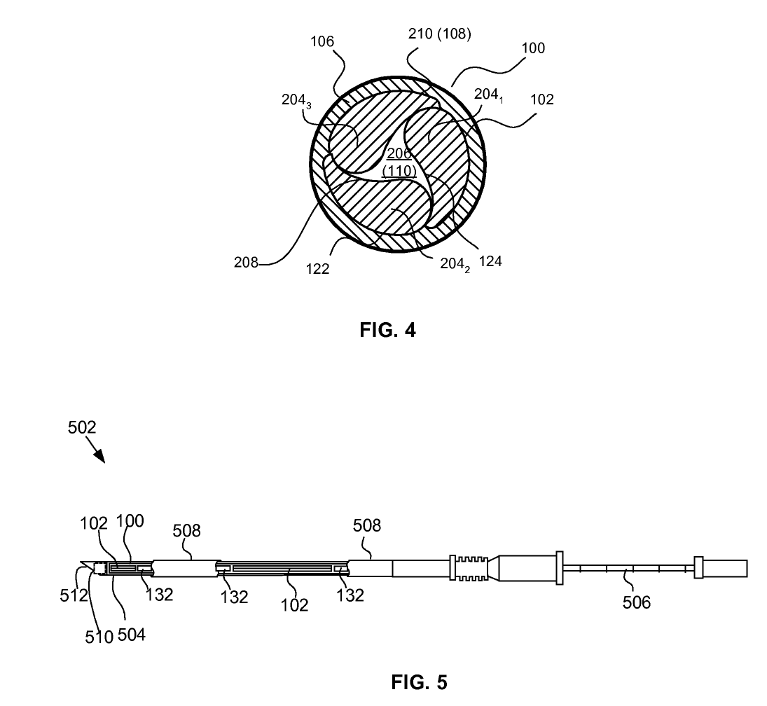

After the strand is manufactured, it can then be inserted into a patient for use in interstitial radiation therapy. An exemplary device that can be used to perform such insertion into a patient will now be described with reference to FIG. 5.

FIG. 5 is a side view of a brachytherapy device 502, which includes a needle 504 and a stylet 506. The needle 504 is shown partially broken away and has a sheath component 508, and is loaded with a strand 100 of the present invention. A beveled end 512 of the needle 504 is plugged with a bio-compatible substance 510 to prevent fluids and tissue from entering the needle 504 and coming in contact with the strand 100 prior to the placement of the strand 100 at its desired location (e.g., adjacent a tumor). The plug 510 can be made out of a bone wax or can be made of one of the bio-absorbable polymers or copolymers listed below. Further the plug 510 can be an end of the strand 100 that is heated and reflowed after the strand is inserted into the needle 504. In operation, the stylet 506 is inserted into the needle 504 until it meets the strand 100. Then the needle 504 is inserted into a patient at the desired site. The strand 100 is gradually extruded from the needle 504 via the static force of the stationary stylet 506, as the needle 504 is pulled back and removed from the patient.

The previous description of the preferred embodiments is provided to enable any person skilled in the art to make or use the embodiments of the present invention. While the invention has been particularly shown and described with reference to preferred embodiments thereof, it will be understood by those skilled in the art that various changes in form and details may be made therein without departing from the spirit and scope of the invention.

What is claimed:

1. A bio-absorbable strand for use in brachytherapy, comprising:
a plurality of discrete hollow bio-absorbable segments spaced apart from one another and encapsulated using a bio-absorbable material to form an elongated member configured to be implantable in patient tissue using a hollow needle;
each hollow bio-absorbable segment having a length, an outer periphery and an inner channel;
radioactive material within at least a portion of the inner channel or coating at least a portion of the outer periphery of each hollow bio-absorbable segment; and
contrast material within at least a portion of the inner channel or coating at least a portion of the outer periphery of each hollow bio-absorbable segment.

2. The strand of claim 1, wherein the plurality of hollow bio-absorbable segments are spaced apart from one another in accordance with a treatment plan such that a spacing between one pair of segments is different than a spacing between another pair of segments.

3. The strand of claim 1, wherein the lengths of the plurality of hollow bio-absorbable segments are in accordance a treatment plan such that a said length of one said segment is different than a length of another said segment.

4. The strand of claim 1, wherein both the radioactive material and the contrast material are within at least a portion of the inner channel each hollow bio-absorbable segment.

5. The strand of claim 1, wherein both the radioactive material and the contrast material coat at least a portion of the outer periphery each hollow bio-absorbable segment.

6. The strand of claim 1, wherein the contrast material comprises a radiopaque material.

7. The strand of claim 1, wherein the plurality of hollow bio-absorbable segments are spaced apart from one another by a plurality of discrete spacers.

8. The strand of claim 1, wherein the encapsulated plurality of discrete hollow bio-absorbable segments are overmolded using the bio-absorbable material to form the elongated member.

9. The strand of claim 1, wherein the encapsulated plurality of discrete hollow bio-absorbable segments are inserted into a hollow tube of the bio-absorbable material to form the elongated member.

10. The strand of claim 1, wherein the encapsulated plurality of discrete hollow bio-absorbable segments are inserted between a pair of bio-absorbable half-shell members of the bio-absorbable material to form the elongated member.

11. The strand of claim 1, further comprising a hollow helical groove extending through at least a portion of the elongated member to improve ultrasound visibility of the elongated member.

12. A bio-absorbable strand for use in brachytherapy, comprising:
a plurality of discrete hollow bio-absorbable segments spaced apart from one another and encapsulated using a bio-absorbable material to form an elongated member configured to be implantable in patient tissue using a hollow needle;
each hollow bio-absorbable segment having a length, an outer periphery and an inner channel;
contrast material within at least a portion of the inner channel of each hollow bio-absorbable segment; and
radioactive material coating at least a portion of the outer periphery of each hollow bio-absorbable segment.

13. The strand of claim 12, wherein the plurality of hollow bio-absorbable segments, which have the contrast material within at least a portion of the inner channel and the radioactive material coating at least a portion of the outer periphery, are spaced apart from one another in accordance with a treatment plan such that a spacing between one pair of segments is different than a spacing between another pair of segments.

14. The strand of claim 12, wherein the lengths of the plurality of hollow bio-absorbable segments, which have the contrast material within at least a portion of the inner channel and the radioactive material coating at least a portion of the outer periphery, are in accordance a treatment plan such that a said length of one said segment is different than a length of another said segment.

15. The strand of claim 12, wherein the contrast material comprises a radiopaque material.

16. The strand of claim 12, wherein the plurality of hollow bio-absorbable segments are spaced apart from one another by a plurality of discrete spacers.

17. The strand of claim 12, wherein the encapsulated plurality of discrete hollow bio-absorbable segments are overmolded using the bio-absorbable material to form the elongated member.

18. The strand of claim 12, wherein the encapsulated plurality of discrete hollow bio-absorbable segments are inserted into a hollow tube of the bio-absorbable material to form the elongated member.

19. The strand of claim 12, wherein the encapsulated plurality of discrete hollow bio-absorbable segments are inserted between a pair of bio-absorbable half-shell members of the bio-absorbable material to form the elongated member.

20. The strand of claim 12, wherein at least one of the outer periphery and the inner channel, of each of the hollow bio-absorbable segments, has a generally helical shape extending along at least a portion of its length to improve ultrasound visibility of each of the segments.

21. The strand of claim 12, further comprising a hollow helical groove extending through at least a portion of the elongated member to improve ultrasound visibility of the elongated member.

22. A bio-absorbable strand for use in brachytherapy, comprising:
a plurality of discrete hollow bio-absorbable segments spaced apart from one another and encapsulated using a bio-absorbable material to form an elongated member configured to be implantable in patient tissue using a hollow needle;
each hollow bio-absorbable segment having a length, an outer periphery and an inner channel;
radioactive material within at least a portion of the inner channel or coating at least a portion of the outer periphery of each hollow bio-absorbable segment; and
contrast material within at least a portion of the inner channel or coating at least a portion of the outer periphery of each hollow bio-absorbable segment;
wherein the plurality of hollow bio-absorbable segments are spaced apart from one another in accordance with a treatment plan such that a spacing between one pair of segments is different than a spacing between another pair of segments.

23. A bio-absorbable strand for use in brachytherapy, comprising:
a plurality of discrete bio-absorbable segments spaced apart from one another and encapsulated using a bio-absorbable material to form an elongated member configured to be implantable in patient tissue using a hollow needle;
each bio-absorbable segment having a length;
radioactive material associated with each bio-absorbable segment; and
contrast material associated with each bio-absorbable segment;
wherein the plurality of bio-absorbable segments are spaced apart from one another in accordance with a treatment plan such that a spacing between one pair of segments is different than a spacing between another pair of segments.
